(19) United States
(12) Patent Application Publication
(10) Pub. No.: US 2011/0189253 A1
(43) Pub. Date: Aug. 4, 2011

(54) BIOMATERIAL COMPOSITION AND METHOD

(75) Inventors: Sean M. Haddock, Memphis, TN (US); Susan J. Drapeau, Cordova, TN (US); Thomas Andrew Simonton, Memphis, TN (US)

(73) Assignee: WARSAW ORTHOPEDIC, INC., Warsaw, IN (US)

(21) Appl. No.: 12/696,124

(22) Filed: Jan. 29, 2010

(51) Int. Cl. A61K 47/32; C08F 2/46; A61K 38/17; A61K 38/18

(52) U.S. Cl. 424/423; 523/113; 522/178; 522/66; 522/65; 522/39; 522/33; 522/48; 514/772; 514/7.6; 514/13.3

(57) ABSTRACT

The disclosure is directed to a composition includes a macromer having a polymeric backbone comprising units with a 1,2-diol or 1,3-diol structure and at least two pendant chains bearing crosslinkable groups, an amphiphilic comonomer, and a crosslinking initiator, wherein the composition has a setting time of less than about 3 minutes. The disclosure is further directed to a kit and a method of making the above-mentioned composition.

### FIELD OF THE DISCLOSURE

This disclosure, in general, relates to a biomaterial composition, a kit and a method of making the composition.

### BACKGROUND

There are many instances in which an appropriate biomaterial is needed for use in repair of tissues and in augmentation of tissues. Applications for an appropriate biomaterial include repair of defects and conditions in a tissue caused by disease, injury, or aging, repair of congenital defects and conditions in a tissue, and augmentation of tissues to provide a desirable functional, reconstructive, or cosmetic change.

WO 01/68721 to BioCure, Inc. discloses a composition useful for tissue bulking that includes macromers having a backbone of a polymer having units with a 1,2-diol and/or 1,3-diol structure. Such polymers include poly(vinyl alcohol) (PVA) and hydrolyzed copolymers of vinyl acetate, for example, copolymers with vinyl chloride or N-vinylpyrrolidone. The backbone polymer contains pendant chains bearing crosslinkable groups and, optionally, other modifiers. The macromers form a hydrogel when crosslinked.

The hydrogel taught in WO 01/68721 is suitable for many bio-applications. However, it does not have the properties necessary for a biomaterial used as an implant in many applications. In particular it does not have setting times necessary for certain applications, for example.

Hence, it would be desirable to provide an improved biomaterial composition.

### SUMMARY

In a particular embodiment, a composition includes a macromer, an amphiphilic comonomer, and a crosslinking initiator. The macromer has a polymeric backbone comprising units with a 1,2-diol or 1,3-diol structure and at least two pendant chains bearing crosslinkable groups. The composition has a setting time of less than about 3 minutes.

In another embodiment, a kit includes a packaged first component and a packaged second component. The packaged first component includes a macromer and an amphiphilic comonomer, the macromer having a polymeric backbone comprising units with a 1,2-diol or 1,3-diol structure and at least two pendant chains bearing crosslinkable groups. The second packaged component includes a crosslinking initiator. The mixed first component and second component have a setting time of less than about 3 minutes.

In another exemplary embodiment, a method of treating a patient with a composition is provided. The method includes mixing a macromer, an amphiphilic comonomer, and a crosslinking initiator, the macromer having a polymeric backbone comprising units with a 1,2-diol or 1,3-diol structure and at least two pendant chains bearing crosslinkable groups. The method includes injecting the composition into a cavity of a patient. The method further includes providing a crosslinked composition with a setting time of less than about 3 minutes.

### DETAILED DESCRIPTION

In an embodiment, a biomaterial composition includes a macromer component, an amphiphilic component, and a crosslinking initiator. The biomaterial composition is a hydrogel composition that has a setting time of less than about 3 minutes. Typically, the biomaterial composition is prepared by homogeneously mixing the macromer component with the amphiphilic component and crosslinking initiator using any suitable mixing method. In an exemplary embodiment, the biomaterial composition is flowable and injectable through a syringe. In another exemplary embodiment, the biomaterial composition can be visually monitored before, during and/or after insertion into an injection site through the use of a dye, contrast agent, or combination thereof.

In an embodiment, the macromers have a backbone of a polymer including units with a 1,2-diol and/or 1,3-diol structure and at least two pendant chains including a crosslinkable group. The macromer backbone can optionally have other pendant chains containing modifiers. In an exemplary embodiment, polyvinyl alcohols (PVAs) can be used as the macromer backbone. Commercially available PVAs include, for example Vinol® 107 from Air Products (MW 22,000 to 31,000, 98 to 98.8% hydrolyzed), Polysciences 4397 (MW 25,000, 98.5% hydrolyzed), BF 14 from Chan Chun, Elvanol® 90-50 from DuPont and UF-120 from Unitika. Other producers are, for example, Nippon Gohsei (Gohsenol®), Monsanto (Gelvatol®), Wacker (Polyviol®), Kuraray, Deriki, and Shin-Etsu. In some cases it is advantageous to use Mowiol® products from Hoechst, in particular those of the 3-83, 4-88, 4-98, 6-88, 6-98, 8-88, 8-98, 10-98, 20-98, 26-88, and 40-88 types.

It is also possible to use copolymers of hydrolyzed or partially hydrolyzed vinyl acetate, which are obtainable, for example, as hydrolyzed ethylene-vinyl acetate (EVA), or vinyl chloride-vinyl acetate, N-vinylpyrrolidone-vinyl acetate, and maleic anhydride-vinyl acetate. If the macromer backbones are, for example, copolymers of vinyl acetate and vinylpyrrolidone, it is again possible to use commercially available copolymers, for example, the commercial products available under the name Luviskol® from BASF. Particular examples include Luviskol VA 37 HM, Luviskol VA 37 E and Luviskol VA 28. If the macromer backbones are polyvinyl acetates, Mowilith 30 from Hoechst is a suitable commercial product.

The PVA typically has a poly(2-hydroxy)ethylene structure. The PVA may also include hydroxy groups in the form of 1,2-glycols. The PVA can be a fully hydrolyzed PVA, with all repeating groups being —CH₂—CH(OH), or a partially hydrolyzed PVA with varying proportions (such as about 1% to about 25%) of pendant ester groups. PVA with pendant ester groups typically have repeating groups of the structure CH₂—CH(OR) where R is COCH₃ group or longer alkyls, with the proviso that the water solubility of the PVA is preserved. The ester groups can also be substituted by acetaldehyde or butyraldehyde acetals that impart a certain degree of hydrophobicity and strength to the PVA. For an application that requires an oxidatively stable PVA, the commercially available PVA can be broken down by NaIO₄—KMnO₄ oxidation to yield a small molecular weight PVA (for example, about 2000 to about 4000).

The PVA is typically prepared by basic or acidic, partial or virtually complete, hydrolysis of polyvinyl acetate. In an embodiment, the PVA includes less than about 50% acetate units, such as less than about 25% of acetate units. In a particular embodiment, amounts of residual acetate units in the PVA, based on the sum of alcohol units and acetate units, are approximately from about 3% to about 25%.

In an embodiment, the PVA has a molecular weight of at least about 2,000. As an upper limit, the PVA may have a molecular weight of up to about 300,000. In an embodiment, the PVA has a molecular weight of up to about 130,000, such as up to about 60,000, or up to about 14,000. In a particular embodiment, the PVA has a molecular weight of about 5,000 to about 200,000. In an exemplary embodiment, the PVA has a molecular weight of about 60,000 to about 150,000. In an exemplary embodiment, the molecular weight of the PVA allows for a biomaterial that is flowable and injectable prior to setting.

Typically, the macromers have at least two pendant chains containing groups that can be crosslinked. “Group” is defined herein to include single polymerizable moieties, such as acrylates, as well as larger crosslinkable regions, such as oligomeric or polymeric regions. The crosslinkers are typically present in an amount of from about 0.01 to about 10 milliequivalents of crosslinker per gram of backbone (meq/g), such as about 0.05 to about 1.5 milliequivalents per gram (meq/g). In an embodiment, the macromers may contain more than one type of crosslinkable group.

The pendant chains are typically attached via the hydroxyl groups of the backbone. In a particular embodiment, the pendant chains having crosslinkable groups that are attached via cyclic acetal linkages to the 1,2-diol or 1,3-diol hydroxyl groups. Crosslinkable groups include, for example, (meth)acrylamide, (meth)acrylate, styryl, vinyl ester, vinyl ketone, vinyl ethers, and the like. In an embodiment, the crosslinkable groups are ethylenically unsaturated functional groups. In a particular embodiment, the crosslinkable groups include olefinically unsaturated groups. In an exemplary embodiment, the crosslinker is N-acryloyl-aminoacetaldehyde dimethylacetal (NAAADA) in an amount from about 6 to about 21 crosslinkers per macromer.

Specific macromers that are suitable for use in the compositions are disclosed in U.S. Pat. Nos. 5,508,317, 5,665,840, 5,807,927, 5,849,841, 5,932,674, 5,939,489, and 6,011,077. The macromers disclosed in U.S. Pat. No. 5,508,317, for example, are PVE prepolymers modified with pendant crosslinkable groups, such as acrylamide groups. containing crosslinkable olefinically unsaturated groups. These macromers can be polymerized by photopolymerization or redox free radical polymerization, for example.

In one embodiment, units containing a crosslinkable group conform, in particular, to the formula I:

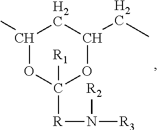

in which R is a linear or branched C₁-C₈ alkylene or a linear or branched C₁-C₁₂ alkane. Suitable alkylene examples include octylene, hexylene, pentylene, butylene, propylene, ethylene, methylene, 2-propylene, 2-butylene and 3-pentylene. In an embodiment, lower alkylene R has up to 6 and in a particular embodiment, up to 4 carbon atoms. In particular embodiment, the groups are ethylene and butylene. Alkanes include, in particular, methane, ethane, n- or isopropane, n-, sec- or tert-butane, n- or isopentane, hexane, heptane, or octane. In an embodiment, groups contain one to four carbon atoms, in particular, one carbon atom.

R₁ is hydrogen, a C₁-C₆ alkyl, or a cycloalkyl, for example, methyl, ethyl, propyl or butyl and R₂ is hydrogen or a C₁-C₆ alkyl, for example, methyl, ethyl, propyl or butyl. In an embodiment, R₁ and R₂ are each hydrogen.

R₃ is an olefinically unsaturated electron attracting copolymerizable radical having up to 25 carbon atoms. In one embodiment, R₃ has the structure:

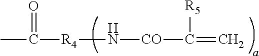

where R₄ is the

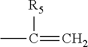

group if n=zero, or the

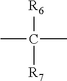

bridge if n=1;

R₅ is hydrogen or C₁-C₄ alkyl, for example, n-butyl, n- or isopropyl, ethyl, or methyl;

n is zero or 1, and in a particular embodiment, zero; and

R₆ and R₇, independently of one another, are hydrogen, a linear or branched C₁-C₈ alkyl, aryl or cyclohexyl, for example, one of the following: octyl, hexyl, pentyl, butyl, propyl, ethyl, methyl, 2-propyl, 2-butyl or 3-pentyl. In an embodiment, R₆ is hydrogen or the CH₃ group, and R₇ is, in a particular embodiment, a C₁-C₄ alkyl group. In an embodiment, R₆ and R₇ are aryl and in a particular embodiment, phenyl.

In another embodiment, R₃ is an olefinically unsaturated acyl group of formula R₈—CO—, in which R₈ is an olefinically unsaturated copolymerizable group having from 2 to 24 carbon atoms, such as from 2 to 8 carbon atoms, or even from 2 to 4 carbon atoms. In a particular embodiment, the olefinically unsaturated copolymerizable radical R₈ having from 2 to 24 carbon atoms is alkenyl having from 2 to 24 carbon atoms, such as alkenyl having from 2 to 8 carbon atoms and or even alkenyl having from 2 to 4 carbon atoms, for example, ethenyl, 2-propenyl, 3-propenyl, 2-butenyl, hexenyl, octenyl or dodecenyl. In a particular embodiment, the groups are ethenyl and 2-propenyl, so that the group —CO—R₈ is the acyl radical of acrylic or methacrylic acid.

In another embodiment, the group R₃ is a radical of formula

—[CO—NH—(R₉—NH—CO—O)_{q}—R₁₀—P]ₚ—CO—R₈

wherein p and q are zero or one and

R₉ and R₁₀ are each independently lower alkylene having from 2 to 8 carbon atoms, arylene having from 6 to 12 carbon atoms, a saturated divalent cycloaliphatic group having from 6 to 10 carbon atoms, arylenealkylene or alkylenearylene having from 7 to 14 carbon atoms or arylenealkylenearylene having from 13 to 16 carbon atoms, and

R₈ is as defined above.

In an embodiment, lower alkylene R₉ or R₁₀ has from 2 to 6 carbon atoms and is straight-chained. Suitable examples include propylene, butylene, hexylene, dimethylethylene and, in a particular embodiment, ethylene.

In a particular embodiment, arylene R₉ or R₁₀ is phenylene that is unsubstituted or is substituted by lower alkyl or lower alkoxy, such as 1,3-phenylene or 1,4-phenylene or methyl-1,4-phenylene.

In a particular embodiment, a saturated divalent cycloaliphatic group R₉ or R₁₀ is cyclohexylene or cyclohexylene-lower alkylene, for example cyclohexylenemethylene, that is unsubstituted or is substituted by one or more methyl groups, such as, for example, trimethylcyclohexylenemethylene, for example the divalent isophorone radical.

In a particular embodiment, the arylene unit of alkylenearylene or arylenealkylene R₉ or R₁₀ is phenylene, unsubstituted or substituted by lower alkyl or lower alkoxy, and the alkylene unit thereof is typically a lower alkylene, such as methylene or ethylene, and in particular, methylene. In an embodiment, the radicals R₉ or R₁₀ are phenylenemethylene or methylenephenylene.

In a particular embodiment, arylenealkylenearylene R₉ or R₁₀ is phenylene-lower alkylene-phenylene having up to 4 carbon atoms in the alkylene unit, for example phenyleneethylenephenylene.

In an embodiment, the groups R₉ and R₁₀ are each independently lower alkylene having from 2 to 6 carbon atoms, phenylene, unsubstituted or substituted by lower alkyl, cyclohexylene or cyclohexylene-lower alkylene, unsubstituted or substituted by lower alkyl, phenylene-lower alkylene, lower alkylene-phenylene or phenylene-lower alkylene-phenylene.

In an embodiment, the group —R₉—NH—CO—O— is present when q is one and absent when q is zero. In a particular embodiment, q is zero.

In an embodiment, the group —CO—NH—(R₉—NH—CO—O)_{q}—R₁₀—O— is present when p is one and absent when p is zero. In a particular embodiment, p is zero.

In an embodiment, macromers wherein p is one, q is zero. In a particular embodiment, macromers wherein p is one, q is zero, and R₁₀ is lower alkylene.

All of the above groups can be monosubstituted or polysubstituted, examples of suitable substituents being the following: C₁-C₄ alkyl, such as methyl, ethyl or propyl, —COOH, —OH, —SH, C₁-C₄ alkoxy (such as methoxy, ethoxy, propoxy, butoxy, or isobutoxy), —NO₂, —NH₂, —NH(C₁-C₄), —NH—CO—NH₂, —N(C₁-C₄ alkyl)₂, phenyl (unsubstituted or substituted by, for example, —OH or halogen, such as Cl, Br or I), —S(C₁-C₄ alkyl), a 5- or 6-membered heterocyclic ring, such as, in particular, indole or imidazole, —NH—C(NH)—NH₂, phenoxyphenyl (unsubstituted or substituted by, for example, —OH or halogen, such as Cl, Br or I), an olefinic group, such as ethylene or vinyl, and CO—NH—C(NH)—NH₂.

In an embodiment, the substituents are lower alkyl, which here, as elsewhere in this description, is C₁-C₄ allyl, C₁-C₄ alkoxy, COOH, SH, —NH₂, —NH(C₁-C₄ alkyl), —N(C₁-C₄ alkyl)₂ or halogen. In a particular embodiment, the substituents are C₁-C₄ alkyl, C₁-C₄ alkoxy, COOH and SH.

For the purposes of this invention, cycloalkyl is, in particular, cycloalkyl, and aryl is, in particular, phenyl, unsubstituted or substituted as described above.

In a particular embodiment, the macromer has a PVA backbone (14 kDa, 17% acetate incorporation) modified with 0.45 meq/g N-acrylamidoacetaldehyde dimethyl acetal (NAAADA) pendant polymerizable groups (about 6.3 crosslinks per chain). In another embodiment, the macromer has a PVA backbone (14 kDa, 17% acetate incorporation) modified with 1.07 meq/g N-acrylamidoacetaldehyde dimethyl acetal (NAAADA) pendant polymerizable groups (about 15 crosslinks per chain).

Modifier Groups

The macromers can include further modifier groups and crosslinkable groups. Some such groups are described in U.S. Pat. Nos. 5,508,317, 5,665,840, 5,807,927, 5,849,841, 5,932,674, 5,939,489, and 6,011,077 and include hydrophobic modifiers such as acetaldehyde diethyl acetal (AADA), butyraldehyde, and acetaldehyde or hydrophilic modifiers such as N-(2,2-dimethoxy-ethyl) succinamic acid, amino acetaldehyde dimethyl acetal, and aminobutyraldehyde dimethyl acetal. These groups may be attached to the macromer backbone, or to other monomeric units included in the backbone. Crosslinkable groups and optional modifier groups can be bonded to the macromer backbone in various ways, for example through a certain percentage of the 1,3-diol units being modified to give a 1,3-dioxane, which contains a crosslinkable group, or a further modifier, in the 2-position. Modifiers include those to modify the hydrophobicity or hydrophilicity, active agents or groups to allow attachment of active agents, photoinitiators, modifiers to enhance or reduce adhesiveness, modifiers to impart thermoresponsiveness, modifiers to impart other types of responsiveness, and additional crosslinking groups.

Attaching a cellular adhesion promoter to the macromers can enhance cellular attachment or adhesiveness of the composition. These agents are well known to those skilled in the art and include carboxymethyl dextran, proteoglycans, collagen, gelatin, glucosaminoglycans, fibronectin, lectins, polycations, and natural or synthetic biological cell adhesion agents such as RGD peptides.

Having pendant ester groups that are substituted by acetaldehyde or butyraldehyde acetals, for example, can increase the hydrophobicity of the macromers and the formed hydrogel. One particularly useful hydrophobic modifying group is acetaldehyde diethyl acetal (AADA) present in an amount from about 0 to 4 milliequivalents per gram (meq/g) of PVA.

Hydrophilic modifiers such as —COOH in the form of N-(2,2-dimethoxy-ethyl) succinamic acid in an amount from about 0 to 2 meq/g PVA can be added to the composition to enhance performance of the composition, such as swelling.

Comonomer

The composition further includes an amphiphilic comonomer. As used herein, the term amphiphilic means that one portion of the molecule is hydrophilic and one portion of the molecule is hydrophobic. In an embodiment, the hydrophilic portion is water soluble and the hydrophobic portion is not water soluble. The monomer as a whole is typically wholly or partially water soluble. Examples of useful amphiphilic comonomers include, but are not limited to, diacetone acrylamide (DAA), N-vinyl caprolactam, N-(butoxymethyl)acrylamide, N-acroyl morpholine, crotonamide, N,N-dimethyl acrylamide, N-octadecylacrylamide, methylene bisacrylamide, polyethylene glycol) diacrylate, acrylamide, and combinations thereof.

When the amphiphilic comonomers are copolymerized with the macromers described above, a hydrogel results that is more cohesive and has higher compressive strength than a hydrogel not containing the amphiphilic comonomer. In a particular embodiment, the comonomer is included in an amount ranging from about 5 to about 95 weight percent, such as about 40 to about 60 weight percent (where weight percent is the percent by weight of the total composition). In a particular embodiment, the amphiphilic comonomer is dicetone acrylamide (DAA) present at about 40 to about 60 weight percent based on the total weight of the composition.

Crosslinking Initiator

The crosslinkable groups of the macromer and amphiphilic comonomer can be crosslinked by any reasonable means. For instance, the ethylenically unsaturated groups of the macromer and comonomer can be crosslinked via free radical initiated polymerization, photoinitiation, redox initiation, chemical initiation, thermal initiation, or any combination thereof. Systems employing these means of initiation are well known to those skilled in the art and may be used in the compositions taught herein.

In an embodiment, a two part redox system is employed. For instance, one part of the system contains a reducing agent. Examples of reducing agents are ferrous salts (such as ferrous gluconate dihydrate, ferrous lactate dihydrate, or ferrous acetate), cuprous salts, cerous salts, cobaltous salts, permanganate, manganous salts, and tertiary amines such as N,N,N,N-tetramethylethylene diamine (TMEDA). The other half of the solution includes an oxidizing agent such as hydrogen peroxide, t-butyl hydroperoxide, t-butyl peroxide, benzoyl peroxide, cumyl peroxide, potassium persulfate, ammonium persulfate, or combination thereof. For instance, examples of a two part redox system include, but are not limited, to ceric ion/nitric acid, ammonium persulphate (APS), N,N,N′,N′-tetramethylethylenediamine (TEMED), benzoyl peroxide, N,N-dimethyl-p-toluidine, hydroquinone, 4-N,N-(dimethylamino)phenethanol, or combinations thereof.

Either or both of the redox solutions can contain macromer, or it may be in a third solution. The solutions containing reductant and oxidant are combined to initiate the crosslinking. In an embodiment, a co-reductant may be used, such as ascorbate, for example, to recycle the reductant and reduce the amount needed. In a particular embodiment, the use of the co-reductant can reduce the toxicity of a ferrous based system.

It may be desirable to include a peroxide stabilizer in redox initiated systems. Examples of peroxide stabilizers are Dequest® products from Solutia Inc., such as for example Dequest® 2010 and Dequest® 2060S. These are phosphonates and chelants that offer stabilization of peroxide systems. Dequest® 2060S is diethylenetriamine penta(methylene phosphonic acid). These can be added in amounts as recommended by the manufacturer.

In an embodiment, the crosslinking initiator is a temperature initiator. In a particular embodiment, the temperature initiator is chosen such that the polymerization reaction occurs at or below normal body temperatures so as not to cause thermal damage to the surgical site or surrounding areas. In an exemplary embodiment, thermal initiation can be accomplished using ammonium persulfate as the crosslinking initiator and optionally using N,N,N,N-tetramethylethylene diamine (TMEDA), which is an amine accelerator.

In an embodiment, the crosslinking initiator may be a chemical initiator. For example, the chemical accelerant may be tetramethylethylenediamine, dimethylaminoethylmethacrylate, ethyl-4-dimethylaminobenzoate, 4-(N,N-dimethylamino)phenethyl alcohol, N,N-3,5-tetramethylaniline, or combinations thereof.

In an embodiment, the crosslinking intiator may be a photoinitator. The photoinitiator may be, for example, ceric ammonium nitrate, N,N-dimethylaminobenzyl alcohol, 4,4′-bis(diethylamino)benzophenone, 2-methyl-1-[4-(hydroxyethoxy)phenyl]-2-methyl-1-propanone, Irgacure 2959 (2-Hydroxy-1-[4-(2-hydroxyethoxy)phenyl]-2-methyl-1-propanone), Irgacure 651 (2-dimethoxy-2-phenyl acetophenone), dimethylaminoethylmethacrylate, ethyl-4-dimethylaminobenzoate, 4-(N,N-dimethylamino)phenethyl alcohol, N,N-3,5-tetramethylaniline, camphorquinone, or combinations thereof.

In an embodiment, the crosslinking initiator is present at an effective amount to initiate crosslinking. The desired amounts of the crosslinking initiator components will be determined by concerns related to gelation speed, toxicity, extent of gelation desired, and stability. In one embodiment, the crosslinking initiator is an applied stimulus, such as light or heat, which causes accelerates crosslinking. In an exemplary embodiment, the effective amount of crosslinking initiator is selected such that the setting time of the biomaterial composition falls within the range of less than about 3 minutes, such as between about 30 seconds to about 3 minutes. In an embodiment, the setting time is less than about 2 minutes, such as between about 30 seconds to about 2 minutes.

Visual Aids

In an exemplary embodiment, the biomaterial composition may include a visual aid such as a dye, a contrast agent, or combination thereof. In an embodiment, when the dye is present, it does not impart any mechanical attributes to the composition. Any reasonable dye is envisioned. In a particular embodiment, the dye or colorant is biocompatible and complies with regulations in 21 C.F.R. parts 70 to 82. Examples include, but are not limited to, FD&C Blue #1, FD&C Blue #2, methylene blue, indocyanine green, and combinations thereof. Typically, the dye is used as an aid to assist the user (for instance, the surgeon, medical technician, aid, or nurse). In an embodiment, the dye can be used to readily inform the surgeon of the type of composition he or she is using. For instance, a purple-colored dye may have become known in the field by users to be indicative of a biomaterial composition suitable for use in the spine, whereas a different color material may be known in the art by users to be indicative of a biomaterial composition suitable for another application.

In an embodiment, the biomaterial composition can be made containing the contrast agent. The contrast agent is a biocompatible material capable of being monitored by any reasonable medical devices, for example, by radiography or magnetic resonance imaging. The contrast agent can be water soluble or water insoluble. Examples of water soluble contrast agents include metrizamide, iopamidol, iothalamate sodium, iodomide sodium, and meglumine. Iodinated liquid contrast agents include, for example, Omnipaque®, Visipaque®, and Hypaque-76®. Examples of water insoluble contrast agents are tantalum, tantalum oxide, barium sulfate, gold, tungsten, and platinum. In a particular embodiment, these are commonly available as particles having a size of about 10 μm or less. Coated-fibers, such as tantalum-coated Dacron fibers can also be used. Other contrast agents include, for example, barium sulfate (BaSO₄), zirconium dioxide, CH_{1S}, Na₂FPO₃, CaF₂, tantalum, and mixtures thereof. In an exemplary embodiment, the contrast agent is barium sulfate. Typically, the barium sulfate contrast agent may be imaged by fluoroscopy. In an embodiment, the barium sulfate is present at an amount sufficient to allow continuous imaging by fluoroscopy during the medical procedure, such as the injection of the composition in a patient, without impacting the mechanical properties or the desired setting time of the composition. Typically, the contrast agent may be selected depending on the medical instrumentation used to view the contrast agent.

The dye, contrast agent, or combination thereof is incorporated temporarily or permanently in the composition. Both solid and liquid dyes and contrast agents can be simply mixed with a solution of the liquid composition prior to crosslinking of the hydrogel. It may also be desirable to include on the macromer a molecule that allows visualization of the formed hydrogel. In an embodiment, dyes and contrast agents are present at an amount sufficient to allow continuous imaging during the medical procedure, such as the injection of the composition in a patient, without impacting the mechanical properties or the desired setting time of the composition. In a particular embodiment, dyes and contrast agents are included in an amount of about 2% to about 30% by weight of the total weight of the composition, such as about 5% to about 15% by weight of the total weight of the composition.

Active Agents and Additives

The biomaterial composition can include an effective amount of one or more biologically or structurally active agents and additives. It may be desirable to deliver the active agent from the formed hydrogel. Active agents that it may be desirable to deliver include prophylactic, therapeutic, diagnostic, and structural agents including organic and inorganic molecules and cells (collectively referred to herein as an “active agent” or “drug”). A wide variety of active agents can be incorporated into the hydrogel. Release of the incorporated additive from the hydrogel may be achieved by diffusion of the agent from the hydrogel, degradation of the hydrogel, and/or degradation of a chemical link coupling the agent to the polymer. In this context, an “effective amount” refers to the amount of active agent required to obtain the desired effect.

Optional active agents include, for example, antibiotics, cytostatis agents, analgesic agents, an antiangiogenic agent, disinfectants, preservatives, growth factors, proliferative factors, proteins, peptides, biopolymers, and mixtures thereof. Other active agents include chemotherapeutic agents, radiation delivery devices, and gene therapy compositions. Examples of active agents that can be incorporated include, but are not limited to, analgesics for the treatment of pain, for example ibuprofen, acetaminophen, and acetylsalicylic acid; antibiotics for the treatment of infection, for example tetracyclines and penicillin and derivatives; and additives for the treatment of infection, for example silver ions, silver (metallic), and copper (metallic). In an exemplary embodiment, the optional active agent includes gentamycine, tobramycine, clindamycine, vancomycine, β-TGF or an analog thereof, and mixtures thereof.

Chemotherapeutic agents that can be incorporated include water soluble chemotherapeutic agents, such as cisplatin (platinol), doxorubicin (adriamycin, rubex), or mitomycin C (mutamycin). Other chermotherapeutic agents include iodinated fatty acid ethyl esters of poppy seed oil, such as lipiodol.

It may be advantageous to incorporate material of biological origin or biological material derived from synthetic methods of manufacture such as proteins, polypeptides, polysaccharides, proteoglycans, and growth factors. In an embodiment, the protein includes a bone morphogenic protein (BMP) series compound.

Cells can be incorporated into the biomaterial composition, including cells to encourage tissue growth or cells to secrete a desired active agent. Cells and tissue that can be incorporated into the composition, including stem cells, autologous nucleus pulposus cells, transplanted autologous nucleus pulposus cells, autologous tissue, fibroblast cells, chondrocyte cells, notochordal cells, allograft tissue and cells, and xenograft tissue and cells.

It may be desirable to include additives to improve the swelling and space-filling properties of the biomaterial, for example, dehydrated spheres, fibers, and the like, hydrophilic polymers, such AMPS, and the like, or hydrocolloids, such as agar, alginates, carboxymethylcellulose, gelatin, guar gum, gum arabic, pectin, starch, and xanthum gum. Incorporation of additives to improve the toughness properties of the injectable biomaterials may prove desirable such as low modulus spheres, fibers, and the like that act as “crack arrestors” and high modulus spheres, fibers, and the like, that act as “reinforcing” agents.

Other additives that may prove advantageous are additives to improve the adhesive properties of the biomaterial, including positively charged polymers, such as Quat, and the like, PVA modified with positive-charged moieties attached to the backbone, cyanoacrylates, PVA modified with cyanoacrylate moieties attached to the backbone, chitosan, urethanes, and mussel-based adhesives.

Active agents and additives can be added to the hydrogel by any reasonable means. Active agents and additives can be incorporated into the composition simply by mixing the agent or additive with the composition prior to administration. The active agent or additive may then be entrapped in the hydrogel that is formed upon administration of the composition. Active agents and additives can be incorporated into preformed articles through encapsulation and other reasonable methods known in the art. The active agent and additives can be in compound form or can be in the form of degradable or nondegradable nano or microspheres. It some cases, it may be possible and desirable to attach the active agent or additives to the macromer. The active agent may be released from the macromer or hydrogel over time or in response to an environmental condition.

### Viscosity Modifier

In a particular embodiment, the composition has a desirable viscosity such that it is injectable and flowable through a needle that is less than about 30 Gauge (Ga). In a particular embodiment, the viscosity of the biomaterial composition prior to setting is, for example, about 20 centipoise (cp) to about 300 cp for delivery of the composition without mechanical assistance or between about 100 cp to about 500 cp for delivery of the composition with mechanical assistance. The viscosity may generally be controlled by the molecular weight of the macromers, the solids content of the solution, and the type and amount of contrast agent present (if any). In an embodiment, the viscosity of the composition may be modified through the use of any suitable viscosity modifier. In an embodiment, the viscosity modifier includes water, saline, blood, a blood product, glycerol, cotton seed oil, poly(ethylene glycol), alkyl glycosides, soybean oil, carbon dioxide, saccharides, electric or magnetic field pulse, or surfactants such as anionic, nonionic, amphoteric/zwitterionic, cationic surfactants, or combinations thereof. The viscosity modifier may be used in any suitable amount to provide a flowable solution as described above.

Methods of Making and Using the Biomaterial

To make the composition, the composition is prepared by mixing the macromer, the amphiphilic comonomer, the crosslinking initiator and any other components and additives, in the desired concentrations for each and proportion to each other. In an embodiment, the reaction product is curable under standard pressure and at a temperature of about 18° C. to about 25° C., such as about 20° C. to about 25° C. Any suitable mixing device may be used for the mixing of the components. In an embodiment, the crosslinking initiator can be mixed with the macromer and comonomer composition before administration, during administration, or after administration to a patient. In a particular embodiment, the mixture may be exposed to conditions to encourage crosslinking and formation of the hydrogel before, during, or after administration of the mixture to a patient. For instance, crosslinking may be initiated by applying stimulus, such as light or heat, when using photoinitiators and thermal initiators.

According to a general method of use, an effective amount of the composition is administered to the desired administration site. The term “effective amount”, as used herein, means the quantity of composition needed to fill the cavity. For instance, the composition is delivered via minimally invasive techniques. In an embodiment, the composition has a desirable viscosity such that it is injectable and flowable through a needle that is less than about 30 Gauge (Ga), such as less than about 25 Ga. The composition is typically flowable through the needle with or without power or pressure assistance. The composition may be administered over any number of treatment sessions. In a particular embodiment, the insertion of the flowable composition may be monitored visually or by medical instrumentation, such as fluoroscopy, until the surgical space has been filled to the desired level.

In a particular embodiment, the composition is injected at the surgical site before substantial crosslinking of the macromers has occurred. This procedure prevents blockage of the syringe needle with gelled polymer. In an exemplary embodiment, the macromer and comonomer are crosslinked into the hydrogel in situ. In addition, in situ or in vivo crosslinking may allow anchoring of the hydrogel to host tissue by covalently bonding with collagen molecules present within the host tissue. In an embodiment, the composition is persistent at the surgical site, typically adhering to the tissue and/or bone at the site. Furthermore, the composition is stable in that it generally does not undergo any significant changes in situ. When set/cured, the composition is also tough and elastic in that it is capable of bearing loads without experiencing undue or permanent deformation. Still further, the composition is believed to be well tolerated by the body in that it produces, at most, tolerable levels of immune and inflammatory responses. It is to be appreciated, however, that in exemplary embodiments of the compositions, while satisfying at least some of these advantages, may not satisfy all of these advantages in every instance.

The biomaterial composition has desirable processing properties. In an embodiment, the composition will set into the formed hydrogel within less than about 3 minutes post initiation of crosslinking. In a particular embodiment, the composition will set into the formed hydrogel within about 30 seconds to about 3 minutes post initiation of crosslinking. In an embodiment, the composition will set into the formed hydrogel within less than about 2 minutes post initiation of crosslinking, such as between about 30 seconds to about 2 minutes post initiation of crosslinking. In certain medical applications, a shorter setting time is desired to decrease the surgeon's waiting time while the composition cures. Accordingly, a lower setting time is beneficial to the patient since it decreases the total time of the medical procedure.

In an exemplary embodiment, the composition advantageously exhibits desirable mechanical properties when crosslinked, i.e. cured. For instance, the composition has advantageous stiffness when cured. In an embodiment, the stiffness of the composition, according to, for instance, ASTM D 2990, ASTM F 2346, ASTM D695, ASTM F 2423, and/or ASTM D 5947, is about 0.1 MPa to about 10 MPa, providing a composition within medical strength regulations and guidelines usable for surgical implants. In a particular embodiment, the stiffness of the composition according to ASTM 2077, Test Methods for Intervertebral Fusion Devices, is about 0.1 MPa to about 10 MPa.

In a particular embodiment, the composition has a combination of desirable properties. For instance, the composition exhibits a desirable viscosity prior to setting and a desirable setting time. In an embodiment, the composition prior to setting has a viscosity of about 20 centipoise to about 300 centipoise with a setting time of less than about 3 minutes. In an embodiment, the composition prior to setting has a viscosity of about 20 centipoise to about 300 centipoise with a setting time of less than about 2 minutes. In a particular embodiment, the composition exhibits a desirable viscosity prior to setting, a desirable setting time, and a visual aid. In an embodiment, the composition exhibits a desirable viscosity prior to setting, a desirable setting time, a visual aid, and a desirable stiffness when crosslinked.

In an embodiment, the composition is sold and distributed to users in a kit where the components are maintained apart (e.g., separately packaged or contained) until they are ready for use in forming the composition. The user may receive a mixer apparatus containing the components in separate compartments thereof. Any suitable apparatus may be used for mixing and delivering the composition's components and mixtures thereof to form the composition. The components likely will be mixed by the user immediately prior to the surgical procedure with a suitable mixing apparatus. In an embodiment, the composition may be prepared in any number of components, such as one-part or more. In a particular embodiment, the biomaterial composition may be prepared as a two-part component system, which form the hydrogel when mixed together. In an embodiment, the composition can be delivered by using a two component system, at least one containing the macromer and comonomer and at least one containing the crosslinking initiator. In a particular embodiment, each separate component of the two component system either contains the reductant or the oxidant. In an exemplary embodiment, the first component contains the macromer and comonomer with the reductant and the second component contains the macromer and comonomer with the oxidant. In an embodiment, the composition may be formed by mixing the first and second components and the composition (or mixture of the components) is delivered by any suitable apparatus to the surgical site before the composition (or mixture) sets and cures.

Potential applications for the biomaterial composition include, but are not limited to, replacement of cartilage found in joints, e.g. knee meniscus, temperomandibular joint, wrist, etc.; vertebroplasty (the augmentation or mechanical support of a compromised vertebrae); bone cement (the adhesive or material used to join bone fragments together); bone filler (the material that fills cavities in bone either permanently or temporarily as new bone fills in the defect); adjunct to metal cages for spinal fixation with screws (the biomaterial may provide additional mechanical support); support of fractures to non-weight bearing bones, e.g. the orbital bones of the face; and prosthetic spinal disc nucleus.

In the embodiment, the biomaterial composition is used for intervertebral disc space. For instance, after creation of a space in the intervertebral disc, if desired, an effective amount of the flowable composition is placed into the space. In an embodiment, the flowable composition is injected into the space, typically under medical visualization. In conjunction with distraction, the biomaterial can be used to restore normal disc height. Once set, the biomaterial can interact with the surrounding tissue at a mechanical property as compression resistant as native intervertebral disc tissue yet typically not harder than the vertebral body.

The above-disclosed subject matter is to be considered illustrative, and not restrictive, and the appended claims are intended to cover all such modifications, enhancements, and other embodiments, which fall within the true scope of the present invention. Thus, to the maximum extent allowed by law, the scope of the present invention is to be determined by the broadest permissible interpretation of the following claims and their equivalents, and shall not be restricted or limited by the foregoing detailed description.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. A composition comprising:
a) a macromer having a polymeric backbone comprising units with a 1,2-diol or 1,3-diol structure and at least two pendant chains bearing crosslinkable groups;
b) an amphiphilic comonomer;
c) a crosslinking initiator;
d) a dye, a contrast agent, or combination thereof;
wherein the composition is flowable prior to setting and has a setting time of less than about 3 minutes.

18. The composition of claim 17, wherein the composition has a setting time of 30 seconds to about 3 minutes.

19. The composition of claim 17, wherein the macromer has a poly(vinyl alcohol) backbone having a molecular weight of between about 5,000 and about 200,000.

20. The composition of claim 17, wherein the amphiphilic comonomer is selected from the group consisting of diacetone acrylamide (DAA), N-vinyl caprolactam, N-(butoxymethyl)acrylamide, N-acroyl morpholine, crotonamide, N,N-dimethyl acrylamide, N-octadecylacrylamide, methylene bisacrylamide, poly(ethylene glycol) diacrylate, acrylamide, and combinations thereof.

21. The composition of claim 20, wherein the comonomer is diacetone acrylamide (DAA) at a concentration between about 40% to about 60% by weight of the total composition.

22. The composition of claim 17, wherein the crosslinking initiator includes a reducing agent and an oxidizing agent, a chemical accelerant, a temperature accelerant, a photoinitiator, or combination thereof.

23. The composition of claim 22, wherein the reducing agent and oxidizing agent includes ceric ion/nitric acid, ammonium persulphate (APS), N,N,N′,N′-tetramethylethylenediamine (TEMED), benzoyl peroxide, N,N-dimethyl-p-toluidine, hydroquinone, 4-N,N-(dimethylamino)phenethanol, or combinations thereof.

24. The composition of claim 22, wherein the chemical accelerant includes tetramethylethylenediamine, dimethylaminoethylmethacrylate, ethyl-4-dimethylaminobenzoate, 4-(N,N-dimethylamino)phenethyl alcohol, N,N-3,5-tetramethylaniline, or combinations thereof.

25. The composition of claim 22, wherein the photoinitiator includes ceric ammonium nitrate, N,N-dimethylaminobenzyl alcohol, 4,4′-bis(diethylamino)benzophenone, 2-methyl-1-[4-(hydroxyethoxy)phenyl]-2-methyl-1-propanone, Irgacure 2959 (2-Hydroxy-1-[4-(2-hydroxyethoxy)phenyl]-2-methyl-1-propanone), Irgacure 651 (2-dimethoxy-2-phenyl acetophenone), dimethylaminoethylmethacrylate, ethyl-4-dimethylaminobenzoate, 4-(N,N-dimethylamino)phenethyl alcohol, N,N-3,5-tetramethylaniline, camphorquinone, or combinations thereof.

26. The composition of claim 17, wherein the contrast agent is selected from the group consisting of barium sulfate (BaSO₄), zirconium dioxide, CHI₃, Na₂FPO₃, CaF₂, tantalum, and mixtures thereof.

27. The composition of claim 17, further comprising a viscosity modifier.

28. The composition of claim 27, wherein the viscosity modifier is water, saline, blood, a blood product, glycerol, cotton seed oil, poly(ethylene glycol), alkyl glycosides, soybean oil, carbon dioxide, saccharides, electric field pulse, magnetic field pulse, anionic surfactants, nonionic surfactants, amphoteric/zwitterionic surfactants, cationic surfactants, or combinations thereof.

29. The composition of claim 17, wherein the composition is injectable through a needle having a size of less than about 30 Gauge prior to setting.

30. The composition of claim 17, having a stiffness of about 0.1 MPa to about 10.0 MPa.

31. The composition of claim 17, further comprising an additive selected from the group consisting of an antibiotic, a cytostatic agent, an analgesic agent, an antiangiogenic agent, a disinfectant, a preservative, a growth factor, a proliferative factor, a protein, a peptide, a biopolymer, a chemotherapeutic, a drug, and mixtures thereof.

32. A kit comprising:
a) a packaged first component comprising a macromer and an amphiphilic comonomer, the macromer having a polymeric backbone comprising units with a 1,2-diol or 1,3-diol structure and at least two pendant chains bearing crosslinkable groups; and
b) a second packaged component comprising a crosslinking initiator;
wherein the mixed first component and second component have a setting time of less than about 3 minutes.

33. The kit of claim 32, wherein the macromer has a poly(vinyl alcohol) backbone having a molecular weight of between about 5,000 and about 200,000.

34. The kit of claim 32, wherein the amphiphilic comonomer is selected from the group consisting of diacetone acrylamide (DAA), N-vinyl caprolactam, N-(butoxymethyl)acrylamide, N-acroyl morpholine, crotonamide, N,N-dimethyl acrylamide, N-octadecylacrylamide, methylene bisacrylamide, poly(ethylene glycol) diacrylate, acrylamide, and combinations thereof.

35. The kit of claim 34, wherein the comonomer is diacetone acrylamide (DAA) at a concentration between about 40% to about 60% by weight of the total composition.

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. A method of treating a patient with a composition, the method comprising:
a) mixing a macromer, an amphiphilic comonomer, and a crosslinking initiator, the macromer having a polymeric backbone comprising units with a 1,2-diol or 1,3-diol structure and at least two pendant chains bearing crosslinkable groups;
b) injecting the composition into a cavity of a patient; and
c) providing a crosslinked composition with a setting time of less than about 3 minutes.

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)
